(19) United States
(12) Patent Application Publication
(10) Pub. No.: US 2018/0353565 A1
(43) Pub. Date: Dec. 13, 2018

(54) PHARMACEUTICAL FORMULATIONS AND THEIR USE FOR THE TREATMENT OF RETINITIS PIGMENTOSA

(71) Applicant: KALEYDE PHARMACEUTICALS AG, Freienwil (CH)

(72) Inventor: Mario DE ROSA, Napoli (IT)

(21) Appl. No.: 16/069,102

(22) PCT Filed: Jan. 11, 2017
(86) PCT No.: PCT/EP2017/050497

§ 371 Date: Jul. 10, 2018

(30) Foreign Application Priority Data
Jan. 12, 2016 (IT) 102016000001989

(51) Int. Cl. A61K 38/07; A61K 38/08; A61K 9/00; A61P 27/02

(52) U.S. Cl. CPC A61K 38/07; A61K 38/08; A61P 27/02; A61K 9/0048; A61K 9/0019

(57) ABSTRACT

Disclosed are pharmaceutical formulations and their use for the treatment of retinitis pigmentosa, comprising tetra- or pentapeptides.

The present invention relates to tetra- or pentapeptides for use in the treatment of retinitis pigmentosa.

### STATE OF THE ART

Retinitis pigmentosa (RP) belongs to a group of hereditary dystrophies characterised by progressive degeneration of the visual cells and abnormalities of the retinal pigment epithelium (RPE) which leads to blindness in a few decades, during which the vision slowly, but inexorably deteriorates.

Night blindness is the first manifestation of the disease, which generally arises during early adolescence, correlated with a deterioration of the rods and followed by progressive death of those cells.

Subsequently, patients suffering from RP exhibit a narrowing of the visual field (tunnel vision), resulting from a further loss of rods in the peripheral part of the retina, where those cells predominate. The disease further develops with a progressive reduction of visual acuity in the central field of vision and alterations of color perception, due to the progressive disappearance of the cones. Although said cells represent less than 5% of all the retinal photoreceptors, their role in the eyesight is crucial, and their degeneration leads to blindness in patients suffering from RP. Further complications of RP are posterior subcapsular cataract and cystoid macular oedema. Other forms of RP also exist: Usher syndrome (wherein RP is associated with deafness); Bardet Biedl syndrome (wherein RP is accompanied by polydactyly, obesity, hypogenitalism and learning disability); and Leber congenital amaurosis (LCA), characterised by blindness from birth.

A distinguishing sign of RP is enormous genetic heterogeneity, with over 3000 mutations (Clin. Genet. 2013, 84, 132-141) in 54 different genes and 61 loci currently known to cause the non-syndromic form of the disease.

The biological mechanisms connecting the mutations responsible for RP with the damage observed in the cones and rods are still not fully understood. Apoptosis is generally considered to be the main cause of photoreceptor death (Curr. Mol. Med. 2009, 9, 375-383; Prog. Retin. Eye Res. 2014, 43, 17-75). The possible causes of cone death include oxidative stress.

Although numerous treatments have been proposed for the treatment of RP, they have all exhibited very limited efficacy so far.

The most extensively studied of the possible treatments is gene therapy. The therapeutic window for gene therapy progressively narrows as the visual cells are lost. Moreover, the disappearance of the photoreceptors shows no sign of declining, even after gene therapy (N. Engl. J. Med. 2015, 372, 1887-1897; Proc. Natl. Acad. Sci. U.S.A 2013, 110, E517-E525), and it is unclear whether this is due to the low doses of viral vectors used to date in humans for safety reasons, or to unknown biological factors.

In addition to the limitations described above, it is clear that gene therapy, being a customized treatment that must take account of the enormous genetic heterogeneity of the disease, is not very practicable, especially due to the very high costs of development for small groups of patients carrying the same mutation.

Another therapeutic approach is to implant various types of electronic prosthesis, which are positioned in contact with the innermost layer of the retina, close to the ganglion cells (epiretinal prostheses), or in place of the photoreceptors (subretinal implants) (Vis. Res. 2002, 42, 393e399; Ophthalmic Res. 2013, 50, 215-220; J. Biomater. Sci. Polym. Ed. 2007, 18, 1031-1055). However, these bionic implants are still rudimentary, and to date only produce a minimal ability to locate light sources, and therefore only improve performance in mobility tests.

Possible pharmacological strategies for treating RP are based on:
neuroprotection (CA2236157, FR2784898, WO2009089399, WO2009111169, WO2007011880);
reduction of oxidative stress (US2015328337, US2014044693, US2012108654, US2008317885, WO2008111497);
inhibition of photoreceptor apoptosis (JP2003089643 JP4953040);
attenuation of retinal inflammation (WO2015110556);
use of antiangiogenics (US2012263794, JP2012062258, US2004176290, U.S. Pat. No. 6,451,799).

Any pharmacological approach to retinal diseases must obviously enable the medicament to cross the physical and functional barriers (eye tissues and blood-retinal barrier) which in practice can prevent the medicament from reaching the target cells in the retina.

The pharmacological strategies proposed to date are based on the use of neurotrophic factors; nerve growth factor (NGF); valproic acid (VPA); vitamin A or docosahexaenoic acid (DHA); anti-inflammatories (dexamethasone, fluocinolone acetonide); anti-oxidants (unoprostone); 9-cis-retinal (QLT091001); antiapoptotics; sphingolipids; and chemical photoswitches.

The multiplicity of therapeutic approaches proposed demonstrates the lack of a really effective treatment for all the many forms of RP.

The advantages, and above all the limitations, of these strategies, were described recently (Progress in Retinal and Eye Research 2015, 48, 62-81).

The information set out above demonstrates the need to develop innovative treatment strategies that allow effective, non-traumatic treatment of RPs of different etiologies, all of which inexorably lead to blindness.

The pharmaceutical formulations for the prevention and treatment of the various forms of RP described below are characterized by limited costs and low-trauma administration routes, which allow repeated administrations and effective, constant levels of active ingredient over time.

### DESCRIPTION OF THE INVENTION

It has now been found that tetra- or pentapeptides, described in WO2008/017372 as cell motility inhibitors and antitumorals, are effective in the treatment of retinitis pigmentosa and the complications thereof, not only by intravitreal administration, but also by systemic, especially subcutaneous, forms of administration.

The object of the invention is therefore said peptides for use in the treatment of retinitis pigmentosa. Said peptides, preferably administered systemically, allow the prevention and treatment of the disease without significant toxic side effects.

The peptides for use according to the invention, which can be used as such or in salified form, have the general formula L₁-X₁-X₂-X₃-X₄, wherein:

L₁ is H, or acyl, or an optionally N-acylated and/or N-alkylated and/or Cα-alkylated amino acid selected from Glu, Gln, Pro, hydroxy-Pro, Azt, Pip, pGlu, Aib, Ac4c, Ac5c and Ac6c;

X₁ and X₃, which can be the same or different, are an optionally N-alkylated and/or Cα-alkylated basic amino acid, selected from Arg, Orn and optionally guanidylated Lys, and phenylalanines substituted at the meta or para positions with an amino or guanidino group;

X₂ is an optionally N-alkylated amino acid selected from Glu, Lys, α-methyl-leucine, α-methyl-valine, α-methyl-glutamic acid, Aib, Ac4c, Ac5c and Ac6c;

X₄ is a hydrophobic amino acid which is amidated or non-amidated at the C-terminal end and optionally Cα-alkylated, selected from Phe, h-Phe, Tyr, Trp, 1-Nal, 2-Nal, h-1-Nal, h-2-Nal, Cha, Chg and Phg.

The following are the conventional abbreviations used for some of the unnatural amino acids which can be included in the formulas of the peptides according to the invention:

Azt=azetidine acid, Pip=pipecolic acid, Aib=α-amino-isobutyric acid, Ac4c=1-aminocyclobutane-1-carboxylic acid, Ac5c=1-aminocyclopentane-1-carboxylic acid, Ac6c=1-aminocyclohexane-1-carboxylic acid, h-Phe=homophenylalanine, 1-Nal=β-1-naphthyl-alanine, 2-Nal=β-2-naphthyl-alanine, h-1-Nal=homo-β-1-naphthyl-alanine, h-2-Nal=homo-β-2-naphthyl-alanine, Cha=cyclohexyl-alanine, Chg=cyclohexyl-glycine, Phg=phenyl-glycine, pGlu=pyroglutamic acid.

The preferred peptides for use according to the invention have the sequences reported in the annexed Sequence Listing and in the table below:

| SEQ ID | L₁ | X₁ | X₂ | X₃ | X₄ |
|---|---|---|---|---|---|
| SEQ ID 1 | Ace | Arg | Glu | Arg | Phe-NH₂ |
| SEQ ID 2 | pGlu | Arg | Glu | Arg | Tyr-OH |
| SEQ ID 3 | Glu | Arg | Glu | Arg | Phe-NH₂ |
| SEQ ID 4 | Ace | Arg | Glu | Arg | Tyr-NH₂ |
| SEQ ID 5 | Ace | Arg | Glu | Arg | Trp-NH₂ |
| SEQ ID 6 | Ace | Arg | Glu | N(Me)Arg | Phe-NH₂ |
| SEQ ID 7 | Ace | Arg | Glu | N(Me)Arg | Tyr-NH₂ |
| SEQ ID 8 | Ace | Arg | Glu | N(Me)Arg | Trp-NH₂ |
| SEQ ID 9 | pGlu | Arg | Glu | N(Me)Arg | Phe-NH₂ |
| SEQ ID 10 | pGlu | Arg | Glu | N(Me)Arg | Tyr-NH₂ |
| SEQ ID 11 | pGlu | Arg | Glu | N(Me)Arg | Trp-NH₂ |
| SEQ ID 12 | pGlu | Arg | Glu | Arg | Phe-NH₂ |
| SEQ ID 13 | pGlu | Arg | Glu | Arg | Tyr-NH₂ |
| SEQ ID 14 | pGlu | Arg | Glu | Arg | Trp-NH₂ |
| SEQ ID 15 | Ace | Arg | Aib | Arg | Phe-NH₂ |
| SEQ ID 16 | Ace | Arg | Aib | Arg | Tyr-NH₂ |
| SEQ ID 17 | Ace | Arg | Aib | Arg | Trp-NH₂ |
| SEQ ID 18 | Ace-Aib | Arg | Aib | Arg | Phe-NH₂ |
| SEQ ID 19 | Ace | Arg | Aib | N(Me)Arg | Phe-NH₂ |
| SEQ ID 20 | Ace | Arg | Aib | N(Me)Arg | Tyr-NH₂ |
| SEQ ID 21 | Ace | Arg | Aib | N(Me)Arg | Trp-NH₂ |
| SEQ ID 22 | pGlu | Arg | Aib | N(Me)Arg | Phe-NH₂ |
| SEQ ID 23 | pGlu | Arg | Aib | N(Me)Arg | Tyr-NH₂ |
| SEQ ID 24 | pGlu | Arg | Aib | N(Me)Arg | Trp-NH₂ |
| SEQ ID 25 | pGlu | Arg | Aib | Arg | Phe-NH₂ |
| SEQ ID 26 | pGlu | Arg | Aib | Arg | Tyr-NH₂ |
| SEQ ID 27 | pGlu | Arg | Aib | Arg | Trp-NH₂ |
| SEQ ID 28 | Ace | Arg | Ac5c | Arg | Phe-NH₂ |
| SEQ ID 29 | Ace | Arg | Ac5c | Arg | Tyr-NH₂ |
| SEQ ID 30 | Ace | Arg | Ac5c | Arg | Trp-NH₂ |
| SEQ ID 31 | Ace | Arg | Ac5c | N(Me)Arg | Phe-NH₂ |
| SEQ ID 32 | Ace | Arg | Ac5c | N(Me)Arg | Tyr-NH₂ |
| SEQ ID 33 | Ace | Arg | Ac5c | N(Me)Arg | Trp-NH₂ |
| SEQ ID 34 | pGlu | Arg | Ac5c | N(Me)Arg | Phe-NH₂ |
| SEQ ID 35 | pGlu | Arg | Ac5c | N(Me)Arg | Tyr-NH₂ |
| SEQ ID 36 | pGlu | Arg | Ac5c | N(Me)Arg | Trp-NH₂ |
| SEQ ID 37 | pGlu | Arg | Ac5c | Arg | Phe-NH₂ |
| SEQ ID 38 | pGlu | Arg | Ac5c | Arg | Tyr-NH₂ |
| SEQ ID 39 | pGlu | Arg | Ac5c | Arg | Trp-NH₂ |
| SEQ ID 40 | Ace | Arg | Glu | Arg | Phe-OH |
| SEQ ID 41 | Ace | Arg | Glu | Arg | Tyr-OH |
| SEQ ID 42 | Ace | Arg | Glu | Arg | Trp-OH |
| SEQ ID 43 | Ace | Arg | Glu | Arg(Me) | Tyr-OH |
| SEQ ID 44 | pGlu | Arg | Glu | Arg(Me) | Phe-OH |
| SEQ ID 45 | pGlu | Arg | Glu | Arg | Trp-OH |
| SEQ ID 46 | Ace | Arg | Aib | Arg | Phe-OH |
| SEQ ID 47 | Ace | Arg | Aib | Arg(Me) | Phe-OH |
| SEQ ID 48 | pGlu | Arg | Aib | Arg(Me) | Tyr-OH |
| SEQ ID 49 | pGlu | Arg | Aib | Arg | Trp-OH |
| SEQ ID 50 | Ace | Arg | Ac5c | Arg | Phe-OH |
| SEQ ID 51 | Ace | Arg | Ac5c | Arg(Me) | Tyr-OH |
| SEQ ID 52 | pGlu | Arg | Ac5c | Arg(Me) | Trp-OH |
| SEQ ID 53 | pGlu | Arg | Ac5c | Arg | Trp-OH |
| SEQ ID 54 | Ace | N(Me)Arg | Aib | Arg | Phe-NH₂ |
| SEQ ID 55 | Ace | N(Me)Arg | Aib | N(Me)Arg | Phe-NH₂ |
| SEQ ID 56 | Ace | Arg | Aib | N(Me)Arg | Phe-NH₂ |
| SEQ ID 57 | Ace | Arg | Aib | Arg | α(Me)Phe-NH₂ |
| SEQ ID 58 | Ace | N(Me)Arg | Aib | Arg | α(Me)Phe-NH₂ |
| SEQ ID 59 | Ace | N(Me)Arg | Aib | N(Me)Arg | α(Me)Phe-NH₂ |
| SEQ ID 60 | Ace | Arg | Aib | N(Me)Arg | α(Me)Phe-NH₂ |
| SEQ ID 61 | Ace-Aib | N(Me)Arg | Aib | Arg | Phe-NH₂ |
| SEQ ID 62 | Ace-Aib | N(Me)Arg | Aib | N(Me)Arg | Phe-NH₂ |
| SEQ ID 63 | Ace-Aib | Arg | Aib | N(Me)Arg | Phe-NH₂ |
| SEQ ID 64 | Ace-Aib | Arg | Aib | Arg | α(Me)Phe-NH₂ |
| SEQ ID 65 | Ace-Aib | N(Me)Arg | Aib | Arg | α(Me)Phe-NH₂ |
| SEQ ID 66 | Ace-Aib | N(Me)Arg | Aib | N(Me)Arg | α(Me)Phe-NH₂ |
| SEQ ID 67 | Ace-Aib | Arg | Aib | N(Me)Arg | α(Me)Phe-NH₂ |

The peptides Ac-Arg-Aib-Arg-α(Me)Phe-NH₂ and Ac-Aib-Arg-Aib-Arg-α(Me)Phe-NH₂ are particularly preferred.

Other types of applications of these peptides, in particular as antitumorals, are known from the literature (FEBS Letters, 582, (2008) 1141-1146. Mol Cancer Ther, 8, (2009) 2708-2717, Mol Cancer Ther, 12, (2013) 1981-1993, Mol Cancer Ther, 13, (2014) 1092-1104). The activity of Ac-Arg-Aib-Arg-α(Me)Phe-NH₂ (SEQ ID 64) (also called UPARANT) in reducing retinal neovascularization in mice with oxygen-induced retinopathy (01R), repairing dysfunctions of the blood-retinal barrier, and reducing the anti-inflammatory markers, when administered by intravitreal injection, is also known (IOVS, (2015), 56(4) 2392-2407).

All the peptides according to the invention are characterised by high affinity for the formyl-peptide receptor (N-formyl-Met-Leu-Phe; FPR) and, by binding to it, exhibit their biological activity. Moreover, although it has been reported that the peptide Ac-Arg-Aib-Arg-α(Me)Phe-NH₂ (SEQ ID 64) (IOVS, (2015), 56(4) 2392-2407) does not modify the structure of the retina, it has even more surprisingly been found that said peptide is able to restore nearly all the strongly deteriorated retinal structure in RCS/KYO rats, one of the most accredited animal models for the study of RP. Finally, despite their peptide nature, the compounds according to the invention exhibit an excellent pharmacological profile and, when administered systemically, especially subcutaneously, cross the blood-eye barrier. In particularly serious cases of RP, intravitreal administration is preferable, and can subsequently be replaced by maintenance treatment comprising systemic administration.

The hydrophilic nature of the peptides according to the invention allows the use of simple, low-cost pharmaceutical formulations which are particularly suitable for injectable formulations for the treatment of RP.

For therapeutic use in the treatment of RP and its various forms, such as Usher syndrome, Bardet Biedl syndrome and Leber congenital amaurosis, as well as its complications, such as posterior subcapsular cataract and cystoid macular oedema, the peptides according to the invention can be formulated as such, or in the form of salts, in liquid or solid pharmaceutical compositions, which can be administered subcutaneously, intramuscularly, intravenously, intraocularly, orally, nasally, sublingually, topically, transdermally or by inhalation, or applied as eyedrops and ointments. Subcutaneous administration is preferred.

The doses of the peptide in humans can vary within wide ranges, typically from 10 μg to 500 mg per dose, and preferably between 1 mg and 200 mg. However, said doses can easily be determined by the expert, depending on the stage of the disease and taking account of the patient's weight, gender and age, and obviously the administration method.

Examples of pharmaceutical compositions of the peptides according to the invention include: a) liquid preparations, such as suspensions, syrups or elixirs for oral, nasal, anal, vaginal or intragastric administration, or for mucosal administration (e.g. perlingual, alveolar or gingival, and via the olfactory or respiratory mucosa); b) sterile solutions, suspensions or emulsions for parenteral, ocular, subcutaneous, intradermal, intramuscular or intravenous administration. In addition to one or more of the peptides according to the invention, said compositions can also contain other active ingredients and rheological compounds commonly used in pharmaceutical technology.

The following examples illustrate the invention in greater detail.

### Example 1—Formulations Containing Ac-Arg-Aib-Arg-α(Me)Phe-NH₂ (SEQ 19) or Ac-Aib-Arg-Aib-Arg-α(Me)Phe-NH₂ (SEQ ID 64)

A) The peptide Ac-Arg-Aib-Arg-α(Me)Phe-NH₂ or Ac-Aib-Arg-Aib-Arg-α(Me)Phe-NH₂ in the form of acetate or succinate salt is dissolved at the active ingredient concentration of 1.2, 7.6 or 16.6 mg/mL in 0.9% aqueous NaCl. The pH is adjusted to 7.2 with an 0.1M solution of NaOH. After sterilization by filtration, the formulation is ready for use.
B) The peptide Ac-Arg-Aib-Arg-α(Me)Phe-NH₂ or Ac-Aib-Arg-Aib-Arg-α(Me)Phe-NH₂ in the form of acetate or succinate salt is dissolved at the active ingredient concentration of 1.2, 7.6 or 16.6 mg/mL in a buffer solution containing: KCl=0.2 g/L; KH₂PO₄=0.24 g/L; NaCl=8.0 g/L; Na₂HPO₄ (anhydrous)=1.44 g/L.

After sterilization by filtration, the formulation is ready for use.

C) The peptide Ac-Arg-Aib-Arg-α(Me)Phe-NH₂ or Ac-Aib-Arg-Aib-Arg-α(Me)Phe-NH₂ in the form of acetate or succinate salt is dissolved at the active ingredient concentration of 1.2, 7.6 or 16.6 mg/mL in a buffer solution containing: CaCl₂) 2H₂O=0.133 g/L; MgCl₂ 6H₂O=0.1 g/L; KCl=0.2 g/L; KH₂PO₄=0.2 g/L; NaCl=8.0 g/L; Na₂HPO₄ (anhydrous)=1.15 g/L. After sterilization by filtration, the formulation is ready for use.
D) An 0.5% solution of sodium hyaluronate (average molecular weight 1200 kDa) in 248.8 mM ammonium acetate (total 50 mL) is washed by dialysis against 24.88 mM ammonium acetate in a membrane with an 8 kDa cutoff. Four washes are conducted: 1×1 L for 7 h; 1×500 mL for 7 h; 2×500 mL against water for 7 h. 505.5 mg of Ac-Arg-Aib-Arg-α(Me)Phe-NH₂ as acetate salt is added to the ammonium hyaluronate solution present in the dialysis membrane (68.5 mL). After stirring for 2 h, the solution is freeze-dried. A salt containing peptide/hyaluronic acid (monomer)/acetate in the ratio 1/1/1 in moles is obtained. Said salt is dissolved in water and the pH adjusted to 7.2 with 0.1M NaOH, until hyaluronic acid concentrations of 14.8 mg/mL, peptide=24.0 mg/mL, are obtained. After sterilization by filtration, the formulation is ready for use.

### Example 2—Efficacy in the Treatment of RCS/KYO Rats by Intravitreal or Subcutaneous Administration

The RCS/Kyo rat (Royal College of Surgeons rat) represents the most commonly used model in the study of this eye disease. RCS rats present retinal degeneration that makes them the ideal model for the study of this disease. In particular, due to deletion in the Mertk gene encoding for a tyrosine kinase receptor, the rats exhibited retinal degeneration from the age of three weeks. The epithelial cells of the retina in these animals are unable to ingest the epithelial photoreceptor cells, and those photoreceptors therefore die.

45 male and female RCS/Kyo rats, aged 14/21 days, were employed, using the tetrapeptide Ac-Arg-Aib-Arg-α(Me)Phe-NH₂, formulated as reported in example 1A, at the concentration of 1.2 mg/mL for intravitreal injection, or 7.6 mg/mL for subcutaneous injection.

The treatment regimen used is set out below:

| | | | | Peptide | | Peptide | |
|---|---|---|---|---|---|---|---|
| | | | | Single dose | | Repeated doses | |
| Number of | | Administration | Type of | Days after | | 6 times a | |
| rats/group | Group | route | treatment | birth | μg | week | μg |
| 10 | S-ivt-sd-t | Intravitreal | Therapeutic | 22 | 4 | — | — |
| 10 | S-sc-rd-p | Subcutaneous | Preventive | 14 | 1800 | 5 weeks | 1800 |
| 10 | S-sc-rd-t | Subcutaneous | Therapeutic | 22 | 1800 | 4 weeks | 1800 |
| 10 | C-sc | Subcutaneous | Control | 14 | Carrier | 5 weeks | Carrier |
| | | | | | only | | only |
| 5 | C-ivt | Intravitreal | Control | 22 | Carrier | — | — |
| | | | | | only | | |

After euthanasia of the rats at the age of 49 days, the thickness of the retina in the various groups was measured under the microscope:

| Study group | Retinal thickness μm | % of control |
|---|---|---|
| S-ivt-sd-t | 132.08 | 110 |
| S-sc-rd-t | 182.67 | 153 |
| S-sc-rd-p | 155.25 | 130 |
| C-sc | 119.75 | 100 |
| C-ivt | 120.00 | 100 |

In all the treated groups, a considerable increase in retinal thickness was observed after both preventive treatment, albeit to a lesser extent, and repeated subcutaneous treatment.

The thickness of the outer layer of granules (outer nuclear layer, ONL, cell body of cones and rods) was also measured.

| Study group | Thickness of ONL μm | % of control |
|---|---|---|
| S-ivt-sd-t | 12.09 | 134 |
| S-sc-rd-t | 17.62 | 196 |
| S-sc-rd-p | 17.64 | 196 |
| C-sc | 9.00 | 100 |
| C-ivt | 9.00 | 100 |

In this case, an increase in the thickness of the ONL was observed for both preventive treatment and therapeutic treatment. A single dose of 4 μg per eye increases the photoreceptor layer by 34%, while repeated subcutaneous doses actually double the thickness of the layer.

### Example 3—Pharmacokinetics and Tissue Distribution in the Rat of Subcutaneous and Intravitreal Administrations of Ac-Arg-Aib-Arg-α(Me)Phe-Nth

10 Male Sprague-Dawley rats were used. The rats were divided into two groups of 5 for determination of plasma pharmacokinetics and tissue distribution, by single subcutaneous administration at the dose of 16.6 mg/kg in a volume of 1 mL/kg. Blood samples were taken after 0.25, 0.5, 1, 2, 4, 6, 8 and 24 hours; subsequently, in the second group of rats, tissue samples were taken at Tmax. The kinetic equation, Cmax, Tmax, plasma half-life and AUC were determined. The concentrations were measured by LC-MS.

The pharmacokinetic parameters are set out in the table below:

| Parameters | Dose 16.6 mg/kg s.c. |
|---|---|
| Kₑₗᵢₘ (h) | 0.309 ± 0.029 |
| T_{1/2} (h) | 2.258 ± 0.226 |
| Cₘₐₓ (μg/mL) | 5.91 ± 1.18 |
| Tₘₐₓ (h) | 2 |
| AUCₗₐₛₜ (μg/h/mL) | 39.29 ± 3.99 |
| AUC_{inf} (μg/h/mL) | 39.33 ± 4.01 |

After the subcutaneous injection a moderate absorption process was observed, as demonstrated by a Tmax of about 2 hours and a Cmax of 5.91±1.18 μg/mL. Said process was followed by disappearance of the compound from the plasma up to 24 hours, albeit with concentration values lower than the sensitivity limit of the method. The kinetic equation can be written as C₍ₜ₎=C₀(e^{-0.32lt}-e^{-0.674t}) if t is expressed in hours and C as mg/L.

This study demonstrates that the subcutaneous administration route makes the compound available. Moreover, as well as reaching the systemic circulation from the injection site, the compound is also present in the various tissues examined, as summarized in the table below:

| Eye | Kidney | Spleen | Brain | Liver | Lung |
|---|---|---|---|---|---|
| 1.068 ± | 16.60 ± | 0.88 ± | 0.12 ± | 4.90 ± | 2.95 ± |
| 0.235 | 3.45 | 0.17 | 0.05 | 1.25 | 0.55 |

The table shows the tissue concentrations expressed as mg/kg at Tmax after administration. As will be seen, the kidney is the organ with the highest values compared with the other tissues. However, in the eyes, which represent the target tissue, the peptide is present with a tissue plasma ratio of about 5.

1. A method of treating retinitis pigmentosa and the complications thereof comprising administering to a subject in need thereof a peptide of general formula L₁-X₁-X₂-X₃-X₄ or salts thereof, wherein:
L₁ is H, or acyl, or an optionally N-acylated and/or N-alkylated and/or Cα-alkylated amino acid selected from Glu, Gln, Pro, hydroxy-Pro, Azt, Pip, pGlu, Aib, Ac3c, Ac4c, Ac5c or Ac6c;
X₁ and X₃, which can be the same or different, are an optionally N-alkylated and/or Cα-alkylated basic amino acid selected from Arg, Orn and optionally guanidylated Lys, and phenylalanines substituted at the meta or para positions with an amino or guanidino group;
X₂ is an optionally N-alkylated amino acid selected from Glu, Lys, α-methyl-leucine, α-methyl-valine, α-methyl-glutamic acid, Aib, Ac3c, Ac4c, Ac5c and Ac6c; and
X₄ is an hydrophobic amino acid, which is amidated or non-amidated at the C-terminal and optionally Cα-alkylated, selected from Phe, h-Phe, Tyr, Trp, 1-Nal, 2-Nal, h-1-Nal, h-2-Nal, Cha, Chg and Phg.

2. The method according to claim 1 wherein the peptide is selected from Ace-Arg-Glu-Arg-Phe-NH₂; pGlu-Arg-Glu-Arg-Tyr-OH; Glu-Arg-Glu-Arg-Phe-NH₂; Ace-Arg-Glu-Arg-Tyr-NH₂; Ace-Arg-Glu-Arg-Trp-NH₂; Ace-Arg-Glu-N(Me)Arg-Phe-NH₂; Ace-Arg-Glu-N(Me)Arg-Tyr-NH₂; Ace-Arg-Glu-N(Me)Arg-Trp-NH₂; pGlu-Arg-Glu-N(Me)Arg-Phe-NH₂; pGlu-Arg-Glu-N(Me)Arg-Tyr-NH₂; pGlu-Arg-Glu-N(Me)Arg-Trp-NH₂; pGlu-Arg-Glu-Arg-Phe-NH₂; pGlu-Arg-Glu-Arg-Tyr-NH₂; pGlu-Arg-Glu-Arg-Trp-NH₂; Ace-Arg-Aib-Arg-Phe-NH₂; Ace-Arg-Aib-Arg-Tyr-NH₂; Ace-Arg-Aib-Arg-Trp-NH₂; Ace-Aib-Arg-Aib-Arg-Phe-NH₂; Ace-Arg-Aib-N(Me)Arg-Phe-NH₂; Ace-Arg-Aib-N(Me)Arg-Tyr-NH₂; Ace-Arg-Aib-N(Me)Arg-Trp-NH₂; pGlu-Arg-Aib-N(Me)Arg-Phe-NH₂; Glu-Arg-Aib-N(Me)Arg-Tyr-NH₂; pGlu-Arg-Aib-N(Me)Arg-Trp-NH₂; pGlu-Arg-Aib-Arg-Phe-NH₂; pGlu-Arg-Aib-Arg-Tyr-NH₂; pGlu-Arg-Aib-Arg-Trp-NH₂; Ace-Arg-Ac5c-Arg-Phe-NH₂; Ace-Arg-Ac5c-Arg-Tyr-NH₂; Ace-Arg-Ac5c-Arg-Trp-NH₂; Ace-Arg-Ac5c-N(Me)Arg-Phe-NH₂; Ace-Arg-Ac5c-N(Me)Arg-Tyr-NH₂; Ace-Arg-Ac5c-N(Me)Arg-Trp-NH₂; pGlu-Arg-Ac5c-N(Me)Arg-Phe-NH₂; pGlu-Arg-Ac5c-N(Me)Arg-Tyr-NH₂; pGlu-Arg-Ac5c-N(Me)Arg-Trp-NH₂; pGlu-Arg-Ac5c-Arg-Phe-NH₂; pGlu-Arg-Ac5c-Arg-Tyr-NH₂; pGlu-Arg-Ac5c-Arg-Trp-NH₂; Ace-Arg-Glu-Arg-Phe-OH; Ace-Arg-Glu-Arg-Tyr-OH; Ace-Arg-Glu-Arg-Trp-OH; Ace-Arg-Glu-N(Me)Arg-Tyr-OH; pGlu-Arg-Glu-N(Me)Arg-Phe-OH; pGlu-Arg-Glu-Arg-Trp-OH; Ace-Arg-Aib-Arg-Phe-OH; Ace-Arg-Aib-N(Me)Arg-Phe-OH; pGlu-Arg-Aib-N(Me)Arg-Tyr-OH; pGlu-Arg-Aib-Arg-Trp-OH; Ace-Arg-Ac5c-Arg-Phe-OH; Ace-Arg-Ac5c-N(Me)Arg-Tyr-OH; pGlu-Arg-Ac5c-N(Me)Arg-Trp-OH; pGlu-Arg-Ac5c-Arg-Trp-OH; Ace-N(Me)Arg-Aib-Arg-Phe-NH₂; Ace-N(Me)Arg-Aib-N(Me)Arg-Phe-NH₂; Ace-Arg-Aib-N(Me)Arg-Phe-NH₂; Ace-Arg-Aib-Arg-α(Me)Phe-NH₂; Ace-N(Me)Arg-Aib-Arg-α(Me)Phe-NH₂; Ace-N(Me)Arg-Aib-N(Me)Arg-α(Me)Phe-NH₂; Ace-Arg-Aib-N(Me)Arg-α(Me)Phe-NH₂; Ace-Aib-N(Me)Arg-Aib-Arg-Phe-NH₂; Ace-Aib-N(Me)Arg-Aib-N(Me)Arg-Phe-NH₂; Ace-Aib-Arg-Aib-N(Me)Arg-Phe-NH₂; Ace-Aib-Arg-Aib-Arg-α(Me)Phe-NH₂; Ace-Aib-N(Me)Arg-Aib-Arg-α(Me)Phe-NH₂; Ace-Aib-N(Me)Arg-Aib-N(Me)Arg-α(Me)Phe-NH₂; Ace-Aib-Arg-Aib-N(Me)Arg-α(Me)Phe-NH₂; and salts thereof.

3. The method according to claim 1 wherein the peptide is selected from Ac-Arg-Aib-Arg-α(Me)Phe-NH₂, Ac-Aib-Arg-Aib-Arg-α(Me)Phe-NH₂, and salts thereof.

4. A pharmaceutical composition comprising one or more peptides according to claim 1.

5. The method according to claim 1 wherein the peptide is administered by subcutaneous, intramuscular, intravenous, intraocular, oral, nasal, sublingual, topical, aerosol or trans-dermal administration, as eye-drops, or as an ocular.

6. The method according to claim 5, wherein the peptide is administered by subcutaneous administration.

7. The method according to claim 5, wherein the peptide is administered intraocular administration.

8. The method according to claim 5, wherein the peptide is administered in a dose of from 10 μg to 500 mg.

9. The method according to claim 6, wherein the peptide is administered in a dose of from 10 μg to 500 mg.

10. The method according to claim 7, wherein the peptide is administered in a dose of from 10 μg to 500 mg.

11. The pharmaceutical composition according to claim 4 comprising of from 10 μg to 500 mg of the peptide.

12. The pharmaceutical composition according to claim 4 further comprising one or more additional active ingredients, carriers, and/or excipients.


SEQUENCE LISTING (65 sequences)

SEQ ID NO: 1 - protein, 5 aa, Artificial Sequence
  synthetic peptide
  MISC_FEATURE (1)..(5)
  MOD_RES (5)..(5): AMIDATION
ERERF

SEQ ID NO: 2 - protein, 4 aa, Artificial Sequence
  synthetic peptide
  MOD_RES (1)..(1): ACETYLATION
  MOD_RES (4)..(4): AMIDATION
RERY

SEQ ID NO: 3 - protein, 4 aa, Artificial Sequence
  synthetic peptide
  MOD_RES (1)..(1): ACETYLATION
  MOD_RES (4)..(4): AMIDATION
RERW

SEQ ID NO: 4 - protein, 4 aa, Artificial Sequence
  synthetic peptide
  MOD_RES (1)..(1): ACETYLATION
  MOD_RES (3)..(3): X is N(Me) Arg
  MOD_RES (4)..(4): AMIDATION
REXF

SEQ ID NO: 5 - protein, 4 aa, Artificial Sequence
  synthetic peptide
  MOD_RES (1)..(1): ACETYLATION
  MOD_RES (3)..(3): X is N(Me)Arg
  MOD_RES (4)..(4): AMIDATION
REXY

SEQ ID NO: 6 - protein, 4 aa, Artificial Sequence
  synthetic peptide
  MOD_RES (1)..(1): ACETYLATION
  MOD_RES (3)..(3): X is N(Me)Arg
  MOD_RES (4)..(4): AMIDATION
REXW

SEQ ID NO: 7 - protein, 5 aa, Artificial Sequence
  synthetic peptide
  MOD_RES (1)..(1): X is pGlu
  MISC_FEATURE (1)..(5)
  MOD_RES (4)..(4): X is N(Me)Arg
  MOD_RES (5)..(5): AMIDATION
XREXF

SEQ ID NO: 8 - protein, 5 aa, Artificial Sequence
  synthetic peptide
  MOD_RES (1)..(1): X is pGlu
  MISC_FEATURE (1)..(5)
  MOD_RES (4)..(4): X is N(Me)Arg
  MOD_RES (5)..(5): AMIDATION
XREXY

SEQ ID NO: 9 - protein, 5 aa, Artificial Sequence
  synthetic peptide
  MOD_RES (1)..(1): X is pGlu
  MISC_FEATURE (1)..(1)
  MISC_FEATURE (4)..(4)
  MOD_RES (4)..(4): Xis N(Me)Arg
  MOD_RES (5)..(5): AMIDATION
XREXW

SEQ ID NO: 10 - protein, 5 aa, Artificial Sequence
  synthetic peptide
  MOD_RES (1)..(1): X is pGlu
  MOD_RES (5)..(5): AMIDATION
XRERF

SEQ ID NO: 11 - protein, 5 aa, Artificial Sequence
  synthetic peptide
  MOD_RES (1)..(1): X is pGlu
  MOD_RES (5)..(5): AMIDATION
XRERY

SEQ ID NO: 12 - protein, 5 aa, Artificial Sequence
  synthetic peptide
  MOD_RES (1)..(1): X is pGlu
  MOD_RES (5)..(5): AMIDATION
XRERW

SEQ ID NO: 13 - protein, 4 aa, Artificial Sequence
  synthetic peptide
  MOD_RES (1)..(1): ACETYLATION
  MOD_RES (2)..(2): Aib
RXRF

SEQ ID NO: 14 - protein, 4 aa, Artificial Sequence
  synthetic peptide
  MOD_RES (1)..(1): ACETYLATION
  MOD_RES (2)..(2): Aib
  MOD_RES (4)..(4): AMIDATION
RXRY

SEQ ID NO: 15 - protein, 4 aa, Artificial Sequence
  synthetic peptide
  MOD_RES (1)..(1): ACETYLATION
  MOD_RES (2)..(2): Aib
  MOD_RES (4)..(4): AMIDATION
RXRW

SEQ ID NO: 16 - protein, 5 aa, Artificial Sequence
  synthetic peptide
  MOD_RES (1)..(1): ACETYLATION
  MOD_RES (1)..(1): Aib
  MOD_RES (3)..(3): Aib
  MOD_RES (5)..(5): AMIDATION
XRXRF

SEQ ID NO: 17 - protein, 4 aa, Artificial Sequence
  synthetic peptide
  MOD_RES (1)..(1): ACETYLATION
  MOD_RES (2)..(2): Aib
  MOD_RES (4)..(4): AMIDATION
RXRF

SEQ ID NO: 18 - protein, 4 aa, Artificial Sequence
  synthetic peptide
  MOD_RES (1)..(1): ACETYLATION
  MOD_RES (2)..(2): Aib
  MOD_RES (3)..(3): X is N(Me)Arg
  MOD_RES (4)..(4): AMIDATION
RXXY

SEQ ID NO: 19 - protein, 4 aa, Artificial Sequence
  synthetic peptide
  MOD_RES (1)..(1): ACETYLATION
  MOD_RES (2)..(2): Aib
  MOD_RES (3)..(3): X is N(Me)Arg
  MOD_RES (4)..(4): AMIDATION
RXXW

SEQ ID NO: 20 - protein, 5 aa, Artificial Sequence
  synthetic peptide
  MOD_RES (1)..(1): X is pGlu
  MOD_RES (3)..(3): Aib
  MOD_RES (4)..(4): X is N(Me)Arg
  MOD_RES (5)..(5): AMIDATION
XRXXF

SEQ ID NO: 21 - protein, 5 aa, Artificial Sequence
  synthetic peptide
  MOD_RES (1)..(1): X is pGlu
  MOD_RES (3)..(3): Aib
  MOD_RES (4)..(4): X is N(Me)Arg
  MOD_RES (5)..(5): AMIDATION
XRXXY

SEQ ID NO: 22 - protein, 5 aa, Artificial Sequence
  synthetic peptide
  MOD_RES (1)..(1): X is pGlu
  MOD_RES (3)..(3): Aib
  MOD_RES (4)..(4): X is N(Me)Aeg
  MOD_RES (5)..(5): AMIDATION
XRXXW

SEQ ID NO: 23 - protein, 5 aa, Artificial Sequence
  synthetic peptide
  MOD_RES (1)..(1): pGlu
  MOD_RES (3)..(3): Aib
  MOD_RES (5)..(5): AMIDATION
XRXRF

SEQ ID NO: 24 - protein, 5 aa, Artificial Sequence
  synthetic peptide
  MOD_RES (1)..(1): pGlu
  MOD_RES (3)..(3): Aib
  MOD_RES (5)..(5): AMIDATION
XRXRY

SEQ ID NO: 25 - protein, 5 aa, Artificial Sequence
  synthetic peptide
  MOD_RES (1)..(1): pGlu
  MOD_RES (3)..(3): Aib
  MOD_RES (5)..(5): AMIDATION
XRXRW

SEQ ID NO: 26 - protein, 4 aa, Artificial Sequence
  synthetic peptide
  MOD_RES (1)..(1): ACETYLATION
  MOD_RES (2)..(2): Ac5c
  MOD_RES (4)..(4): AMIDATION
RXRF

SEQ ID NO: 27 - protein, 4 aa, Artificial Sequence
  synthetic peptide
  MOD_RES (1)..(1): ACETYLATION
  MOD_RES (2)..(2): Ac5c
  MOD_RES (4)..(4): AMIDATION
RXRY

SEQ ID NO: 28 - protein, 4 aa, Artificial Sequence
  synthetic peptide
  MOD_RES (1)..(1): ACETYLATION
  MOD_RES (2)..(2): Ac5c
  MOD_RES (4)..(4): AMIDATION
RXRW

SEQ ID NO: 29 - protein, 4 aa, Artificial Sequence
  synthetic peptide
  MOD_RES (1)..(1): ACETYLATION
  MOD_RES (2)..(2): Ac5c
  MOD_RES (3)..(3): N(Me)Arg
  MOD_RES (4)..(4): AMIDATION
RXXF

SEQ ID NO: 30 - protein, 4 aa, Artificial Sequence
  synthetic peptide
  MOD_RES (1)..(1): ACETYLATION
  MOD_RES (2)..(2): Ac5c
  MOD_RES (3)..(3): N(Me)Arg
  MOD_RES (4)..(4): AMIDATION
RXXY

SEQ ID NO: 31 - protein, 4 aa, Artificial Sequence
  synthetic peptide
  MOD_RES (1)..(1): ACETYLATION
  MOD_RES (2)..(2): Ac5c
  MOD_RES (3)..(3): N(Me)Arg
  MOD_RES (4)..(4): AMIDATION
RXXW

SEQ ID NO: 32 - protein, 5 aa, Artificial Sequence
  synthetic peptide
  MOD_RES (1)..(1): pGlu
  MOD_RES (3)..(3): Ac5c
  MOD_RES (4)..(4): N(Me)Arg
  MOD_RES (5)..(5): AMIDATION
XRXXF

SEQ ID NO: 33 - protein, 5 aa, Artificial Sequence
  synthetic peptide
  MOD_RES (1)..(1): ACETYLATION
  MOD_RES (1)..(1): ACETYLATION
  MOD_RES (1)..(1): Xaa is pGlu
  MOD_RES (3)..(3): Ac5c
  MOD_RES (4)..(4): N(Me)Arg
  MOD_RES (5)..(5): AMIDATION
XRXXY

SEQ ID NO: 34 - protein, 5 aa, Artificial Sequence
  synthetic peptide
  MOD_RES (1)..(1): pGlu
  MOD_RES (3)..(3): Ac5c
  MOD_RES (4)..(4): N(Me)Arg
  MOD_RES (5)..(5): AMIDATION
XRXXW

SEQ ID NO: 35 - protein, 5 aa, Artificial Sequence
  synthetic peptide
  MOD_RES (1)..(1): pGlu
  MOD_RES (3)..(3): Ac5c
  MOD_RES (5)..(5): AMIDATION
XRXRF

SEQ ID NO: 36 - protein, 5 aa, Artificial Sequence
  synthetic peptide
  MOD_RES (1)..(1): pGlu
  MOD_RES (3)..(3): Ac5c
  MOD_RES (5)..(5): AMIDATION
XRXRY

SEQ ID NO: 37 - protein, 5 aa, Artificial Sequence
  synthetic peptide
  MOD_RES (1)..(1): pGlu
  MOD_RES (3)..(3): Ac5c
  MOD_RES (5)..(5): AMIDATION
XRXRW

SEQ ID NO: 38 - protein, 4 aa, Artificial Sequence
  synthetic peptide
  MOD_RES (1)..(1): ACETYLATION
RERF

SEQ ID NO: 39 - protein, 4 aa, Artificial Sequence
  synthetic peptide
  MOD_RES (1)..(1): ACETYLATION
RERY

SEQ ID NO: 40 - protein, 4 aa, Artificial Sequence
  synthetic peptide
  MOD_RES (1)..(1): ACETYLATION
RERW

SEQ ID NO: 41 - protein, 4 aa, Artificial Sequence
  synthetic peptide
  MOD_RES (1)..(1): ACETYLATION
  MOD_RES (3)..(3): N(Me)Arg
REXY

SEQ ID NO: 42 - protein, 5 aa, Artificial Sequence
  synthetic peptide
  MOD_RES (1)..(1): pGlu
  MOD_RES (4)..(4): Arg(Me)
XREXF

SEQ ID NO: 43 - protein, 5 aa, Artificial Sequence
  synthetic peptide
  MOD_RES (1)..(1): pGlu
XRERW

SEQ ID NO: 44 - protein, 4 aa, Artificial Sequence
  synthetic peptide
  MOD_RES (1)..(1): ACETYLATION
  MOD_RES (2)..(2): Aib
  MOD_RES (3)..(3): Arg(Me)
RXXF

SEQ ID NO: 45 - protein, 4 aa, Artificial Sequence
  synthetic peptide
  MOD_RES (1)..(1): ACETYLATION
  MOD_RES (2)..(2): Aib
  MOD_RES (3)..(3): Arg(Me)
RXXF

SEQ ID NO: 46 - protein, 5 aa, Artificial Sequence
  synthetic peptide
  MOD_RES (1)..(1): pGlu
  MOD_RES (3)..(3): Aib
  MOD_RES (4)..(4): Arg(Me)
XRXXY

SEQ ID NO: 47 - protein, 5 aa, Artificial Sequence
  synthetic peptide
  MOD_RES (1)..(1): pGlu
  MOD_RES (3)..(3): Aib
XRXRW

SEQ ID NO: 48 - protein, 4 aa, Artificial Sequence
  synthetic peptide
  MOD_RES (1)..(1): ACETYLATION
  MOD_RES (2)..(2): Ac5c
RXRF

SEQ ID NO: 49 - protein, 4 aa, Artificial Sequence
  synthetic peptide
  MOD_RES (1)..(1): ACETYLATION
  MOD_RES (2)..(2): Ac5c
  MOD_RES (3)..(3): Arg(Me)
RXXY

SEQ ID NO: 50 - protein, 5 aa, Artificial Sequence
  synthetic peptide
  MOD_RES (1)..(1): pGlu
  MOD_RES (3)..(3): Ac5c
  MOD_RES (4)..(4): Arg(Me)
XRXXW

SEQ ID NO: 51 - protein, 5 aa, Artificial Sequence
  synthetic peptide
  MOD_RES (1)..(1): pGlu
  MOD_RES (3)..(3): Ac5c
XRXRW

SEQ ID NO: 52 - protein, 4 aa, Artificial Sequence
  synthetic peptide
  MOD_RES (1)..(1): ACETYLATION
  MOD_RES (1)..(1): N(Me)Arg
  MOD_RES (2)..(2): Aib
  MOD_RES (4)..(4): AMIDATION
XXRF

SEQ ID NO: 53 - protein, 4 aa, Artificial Sequence
  synthetic peptide
  MOD_RES (1)..(1): ACETYLATION
  MOD_RES (1)..(1): N(Me)Arg
  MOD_RES (2)..(2): Aib
  MOD_RES (3)..(3): N(Me)Arg
  MOD_RES (4)..(4): AMIDATION
XXXF

SEQ ID NO: 54 - protein, 4 aa, Artificial Sequence
  synthetic peptide
  MOD_RES (1)..(1): ACETYLATION
  MOD_RES (2)..(2): Aib
  MOD_RES (3)..(3): N(Me)Arg
  MOD_RES (4)..(4): AMIDATION
RXXF

SEQ ID NO: 55 - protein, 4 aa, Artificial Sequence
  synthetic peptide
  MOD_RES (1)..(1): ACETYLATION
  MOD_RES (2)..(2): Aib
  MOD_RES (4)..(4): alpha(Me)Phe
  MOD_RES (4)..(4): AMIDATION
RXRX

SEQ ID NO: 56 - protein, 4 aa, Artificial Sequence
  synthetic peptide
  MOD_RES (1)..(1): ACETYLATION
  MOD_RES (1)..(1): N(Me)Arg
  MOD_RES (2)..(2): Aib
  MOD_RES (3)..(3): N(Me)Arg
  MOD_RES (4)..(4): alpha.(Me)Phe
  MOD_RES (4)..(4): AMIDATION
XXXX

SEQ ID NO: 57 - protein, 4 aa, Artificial Sequence
  synthetic peptide
  MOD_RES (1)..(1): ACETYLATION
  MOD_RES (1)..(1): N(Me)Arg
  MOD_RES (2)..(2): Aib
  MOD_RES (3)..(3): N(Me)Arg
  MOD_RES (4)..(4): alpha(Me)Phe
  MOD_RES (4)..(4): AMIDATION
XXXX

SEQ ID NO: 58 - protein, 4 aa, Artificial Sequence
  synthetic peptide
  MOD_RES (1)..(1): ACETYLATION
  MOD_RES (2)..(2): Aib
  MOD_RES (3)..(3): N(Me)Arg
  MOD_RES (4)..(4): alpha(Me)Phe
  MOD_RES (4)..(4): AMIDATION
RXXX

SEQ ID NO: 59 - protein, 5 aa, Artificial Sequence
  synthetic peptide
  MOD_RES (1)..(1): ACETYLATION
  MOD_RES (1)..(1): Aib
  MOD_RES (2)..(2): N(Me)Arg
  MOD_RES (3)..(3): Aib
  MOD_RES (5)..(5): AMIDATION
XXXRF

SEQ ID NO: 60 - protein, 5 aa, Artificial Sequence
  synthetic peptide
  MOD_RES (1)..(1): ACETYLATION
  MOD_RES (1)..(1): Aib
  MOD_RES (2)..(2): N(Me)Arg
  MOD_RES (3)..(3): Aib
  MOD_RES (4)..(4): N(Me)Arg
  MOD_RES (5)..(5): AMIDATION
XXXXF

SEQ ID NO: 61 - protein, 5 aa, Artificial Sequence
  synthetic peptide
  MOD_RES (1)..(1): ACETYLATION
  MOD_RES (1)..(1): Aib
  MOD_RES (3)..(3): Aib
  MOD_RES (4)..(4): N(Me)Arg
  MOD_RES (5)..(5): AMIDATION
XRXXF

SEQ ID NO: 62 - protein, 5 aa, Artificial Sequence
  synthetic peptide
  MOD_RES (1)..(1): ACETYLATION
  MISC_FEATURE (1)..(5)
  MOD_RES (1)..(1): Aib
  MOD_RES (3)..(3): Aib
  MOD_RES (5)..(5): alpha(Me)Phe
  MOD_RES (5)..(5): AMIDATION
XRXRX

SEQ ID NO: 63 - protein, 5 aa, Artificial Sequence
  synthetic peptide
  MOD_RES (1)..(1): ACETYLATION
  MOD_RES (1)..(1): Aib
  MOD_RES (2)..(2): N(Me)Arg
  MOD_RES (3)..(3): Aib
  MOD_RES (5)..(5): alpha(Me)Phe
  MOD_RES (5)..(5): AMIDATION
XXXRX

SEQ ID NO: 64 - protein, 5 aa, Artificial Sequence
  synthetic peptide
  MOD_RES (1)..(1): ACETYLATION
  MOD_RES (1)..(1): Aib
  MOD_RES (2)..(2): N(Me)Arg
  MOD_RES (3)..(3): Aib
  MOD_RES (4)..(4): N(Me)Arg
  MOD_RES (5)..(5): alpha(Me)Phe
  MOD_RES (5)..(5): AMIDATION
XXXXX

SEQ ID NO: 65 - protein, 5 aa, Artificial Sequence
  synthetic peptide
  MOD_RES (1)..(1): ACETYLATION
  MOD_RES (1)..(1): Aib
  MOD_RES (3)..(3): Aib
  MOD_RES (4)..(4): N(Me)Arg
  MOD_RES (5)..(5): alpha(Me)Phe
  MOD_RES (5)..(5): AMIDATION
XRXXX